(19) United States
(12) Patent Application Publication
(10) Pub. No.: US 2019/0298568 A1
(43) Pub. Date: Oct. 3, 2019

(54) WEARABLE REHABILITATION AND RELAXATION AID

(71) Applicant: Jarviel Rayford, SR., Apple Valley, CA (US)

(72) Inventor: Jarviel Rayford, SR., Apple Valley, CA (US)

(21) Appl. No.: 15/679,012

(22) Filed: Oct. 20, 2017

Related U.S. Application Data
(60) Provisional application No. 62/512,077, filed on May 29, 2017.

(51) Int. Cl. A61F 7/02; A61F 5/01

(52) U.S. Cl. CPC A61F 7/02; A61F 5/0102; A61F 2007/0042; A61F 2007/023; A61H 2201/165

(57) ABSTRACT

A wearable rehabilitation and relaxation aid is configured to provide support to an injured area. The wearable rehabilitation and relaxation aid has a pack that is covered by a breathable and washable material. A strap is joined to the breathable and washable material with triple stitching in order to fix the wearable rehabilitation and relaxation aid to the injured area.

### BACKGROUND OF THE INVENTION

The present invention relates to rehabilitation and, more particularly, to a wearable rehabilitation and relaxation aid.

Systems currently used are either limited, or assisted by other applications of bandages, wraps, tapes or braces, they are big and bulky, can be of a onetime usage and messy after melting. The current systems provide inadequate coverage for the supportive muscles in conjunction with the injured area. The injured individual is subject to be exposed to the outside elements which could compound the situation. Finally, the functionality is primitive and obsolete with the diversified area of compound injuries worldwide.

As can be seen, there is a need for a wearable rehabilitation and relaxation aid.

### BRIEF DESCRIPTION OF THE DRAWINGS

FIG. 1 is a perspective view of an embodiment of the present invention;

FIG. 2 is an exploded view of an embodiment of the present invention;

FIG. 3 is a section view taken along line 3-3 in FIG. 1;

FIG. 4 is a perspective view of an embodiment of the present invention;

FIG. 5 is a perspective view of an embodiment of the present invention; and

FIG. 6 is a flow chart of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes an enclosed hot/cold compress therapy for the treatment of acute and chronic injuries i.e. muscle bruises or tears, tissue, tendon, or cartilage contusions, pre-surgery or post surgery, carpal tunnel syndrome, heat exhaustion or utilized recreationally for additional relax therapy. These reusable therapy inserts are composed of an Elastogel material, that is flexible, latex free, does not leak or dry out if punctured, also removably applied to the injured area of the body. Enclosed within the breathable, washable material the gel packs are held in place by counter compression technology straps, which improves comfort by reducing exposure to the outside elements. Also, the present invention allows full range of motion with limited or no restriction while maintaining the anatomical structure of application to the injured person. These diverse designs assist with the reoccurrence of commonly reported injuries to be properly addressed.

The present invention provides a protective barrier from the hot/cold packs. The present invention provides comfort, conformability and element control from the outside elements with reusable and washable material with counter compression technology wraps. The material that is used may be Elatogel, which is latex free, tough, and flexible and does not leak if punctured, microwavable and easy to use. The present invention addresses the general and reoccurring reported injuries i.e.; ankle back, knee, and shoulder for example. The present invention also includes designs that address specific injuries i.e.; hamstring, quadriceps, carpal tunnel syndrome, ribs, calf for example.

The present invention includes a breathable and washable material, which is the protective barrier between the Elastogel packs and the protective barrier from the outside elements. The Elastogel inserts are placed as the injured individual requests, and have an added advantage for coverage for the supportive muscles and the injured area. The Counter Compression Technology Velcro/wraps are designed to provide an added support for the hot/cold pack inserts that is added to the injured area or utilized for therapeutic purposes.

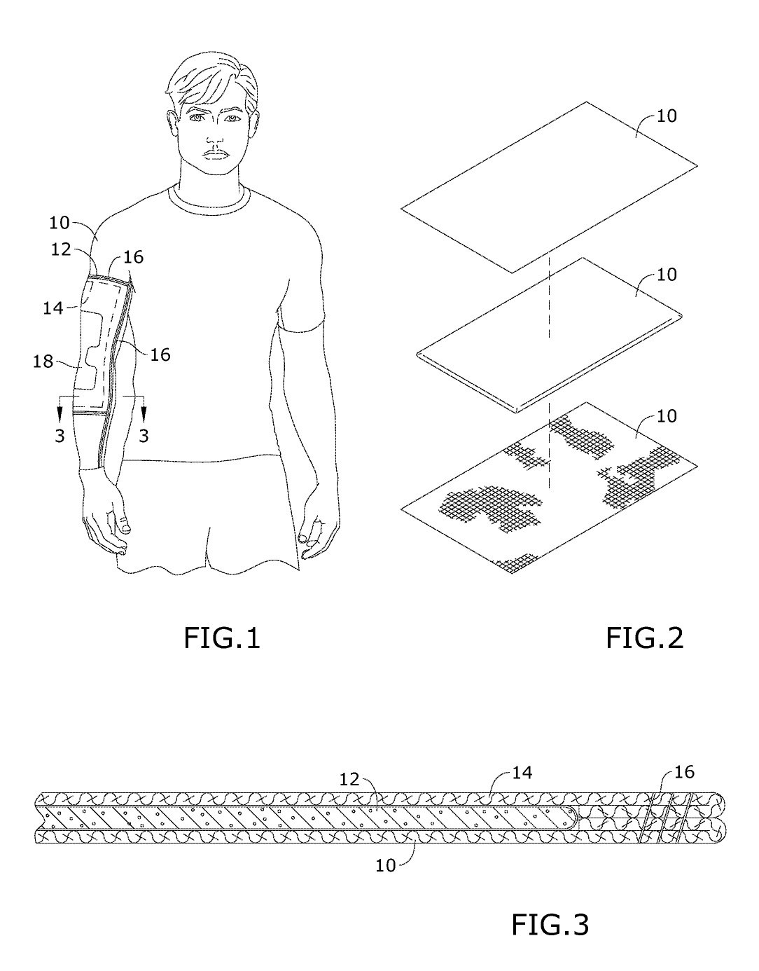

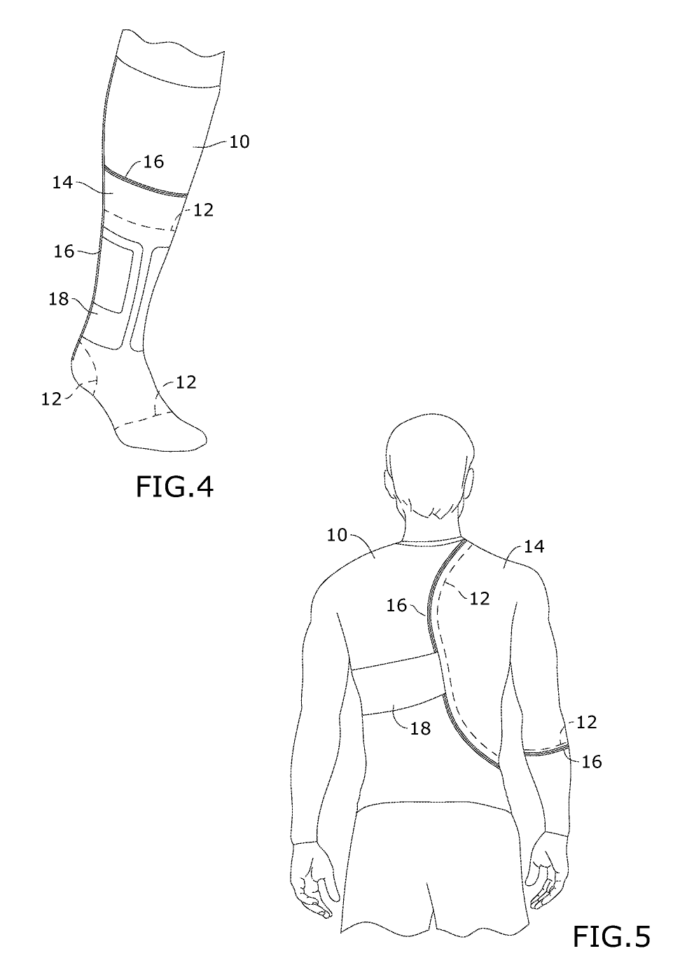

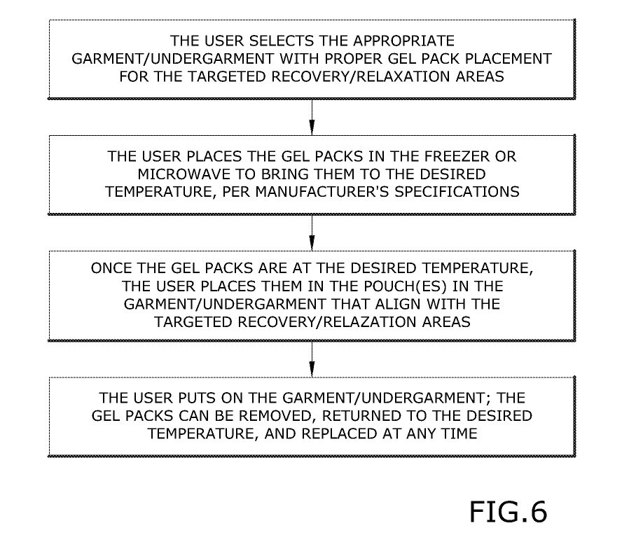

Referring now to FIGS. 1 through 6, the present invention may include the following components:
10: breathable and washable material
12: the hot or cold pack
14: the counter-compression technology
16: triple stitching
18: hook and look wrap
20: exemplary use

The Breathable/Washable Material 10 serves as the skin and protective barrier to hold everything together. The Elastogel packs 12 for the general and injury specific designs, specific placement in conjunction with the protective barrier is the bulk of my invention and what makes it unique to the injured individual. The Counter Compression Technology wrap/straps 14 serves as the secondary support and applied comfort and security to the injured individual using medical application of “RICE”. Finally, the stitching 16 is the glue to hold everything in place free flowing nonrestrictive, even after multiple washings, applications, sever weather, and conditions.

The breathable and washable material provides the injured or rehabilitating individual the initial source of compression, comfort and protective barrier used in the rehabilitative world of “RICE”. The newly added protective barrier between the Elastogel packs is also a barrier from the outside elements. Elastogel inserts within the breathable material are designed and placed as the general or injury specific designs upon individual requests. Enclosed hot/cold compress therapy for the treatment of acute and chronic injuries i.e. muscle bruises or tears, tissue, tendon, or cartilage contusions, pre-surgery or post surgery, carpal tunnel syndrome, heat exhaustion or utilized recreationally for additional relaxation therapy. The Counter Compression Technology Velcro/wraps are designed to provide an added support for the hot/cold pack inserts that is added to the injured area or utilized for therapeutic purposes. Finally, the stitching brings these elements together and hold in place with strength and comfort.

A method of using the present invention may include the following. The user places the gel pack(s) in the freezer or microwave for desired temperature. Then the places the breathable material of their body. These reusable therapy inserts may be composed of an Elastogel material, that is flexible, latex free, does not leak or dry out if punctured. Enclosed within a breathable, washable material and held snug with counter compression technology straps, which reduces exposure to the outside elements, allowing full range of motion with limited or no restriction while maintaining the anatomical structure of application to the injured person. These diverse designs allow the reoccurrence of common injuries to be properly addressed, also within the world-wide scope of unique injuries there are also specific designs for these applications for the injured being.

It should be understood, of course, that the foregoing relates to exemplary embodiments of the invention and that modifications may be made without departing from the spirit and scope of the invention as set forth in the following claims.

What is claimed is:

1. A wearable rehabilitation and relaxation aid as described above and illustrated in the Figures.
